(19) United States
(12) Patent Application Publication
(10) Pub. No.: US 2009/0312824 A1
(43) Pub. Date: Dec. 17, 2009

(54) Method and Apparatus for Influencing the Temperature of an Area of the Body

(76) Inventors: Layne Matthew Dean, Nederland, TX (US); Mechelle D. Dean, Nederland, TX (US)

Correspondence Address: KEELING PATENTS AND TRADEMARKS, LLC, 3310 KATY FREEWAY, SUITE 100, HOUSTON, TX, 77007, US

(21) Appl. No.: 12/137,120

(22) Filed: Jun. 11, 2008

(51) Int. Cl. A61F 7/00

(52) U.S. Cl. 607/109

(57) ABSTRACT

An exemplary embodiment of the invention comprises a device and method for altering the temperature of an area of an individual's body. This alteration may be accomplished by cooling or heating the skin proximate the area of the body, including but not limited to the area proximate the carotid artery, by utilizing the embodiments of the invention disclosed and alluded to herein. An embodiment of the invention generally comprises a container, a container cap connectable thereto, at least one inlet tube, at least one outlet tube, and a temperature-altering element contained in an operable configuration in relation to the container. An exemplary method of use includes the steps of operably coupling a temperature-altering element with a container; introducing fluid into the interior space of the container; altering the temperature of the fluid; and expelling the altered fluid toward a body surface.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Field of the Invention

The embodiments of the invention relates generally to the manipulation of the temperature of at least one area of the body of a living being. More particularly, the embodiments of the invention relates to a method and apparatus for cooling or heating the blood flowing to the brain through the carotid arteries without the necessity of an invasive procedure.

2. Description of the Related Art

There are several different types of temperature-related illnesses. These illnesses can range from fairly minor and temporary discomfort to potentially life-threatening situations. Heat-related illnesses are caused by a variety of factors some of which include extreme heat, high humidity, dehydration and vigorous exercise. Cold-related illnesses occur when an individual loses heat faster than they can produce it. It is a common misconception that illnesses such as hypothermia occur only at very cold temperatures. In fact, hypothermia may occur at cool temperatures in response to an individual becoming chilled from such common occurrences as getting wet in the rain, from sweating, or from submerging themselves in cold water.

Several internal temperature-manipulating inventions are currently known. U.S. Pat. No. 7,311,725, issued to John D. Dobak, III on Dec. 25, 2007, discloses an invasive device and method for cooling the blood flow in a vena cava whereby the cooled blood is then distributed throughout the body. A heat transfer element is employed intravascularly in order to systemically cool the blood flowing through the vasculature. This type of procedure cannot be performed at the onset of a temperature-related illness unless such illness occurs within a facility equipped to perform such procedures. The majority of temperature-related illnesses occur in any number of locations, the majority of which are not within these types of facilities. The embodiments of the invention disclosed herein may be utilized at any location.

U.S. Pat. No. 6,923,826, issued to Donald J. Larnard and Dan Sachs on Aug. 2, 2005, discloses a non-systemic approach to affecting the temperature of a portion of the body utilizing a cooling device that is placed through the skull in order to effect the desired cooling of the brain. Again, this procedure is not possible to perform during the onset of the majority of temperature-related illnesses and must be performed in a facility equipped to handle such a procedure.

U.S. Pat. No. 6,156,057, issued to James Allan Fox on Dec. 5, 2000, discloses a device that is inserted either through the nasal cavity to apply heat to the nasal passage, sinus and/or hypothalamus in order to induce a physiological reaction that will trigger a compensatory cooling response within the animal. The device may also be inserted through an orifice created in the skull whereby the device is directed to the hypothalamic region of the brain in order to induce the compensatory cooling response. The later method must be performed in a facility equipped to provide such invasive care. The former method requires time in order to have the individual's system react to the heating of the desired area in order to produce a cooling effect in response.

U.S. Pat. No. 3,776,241, issued to James H. Magilton and Curran S. Swift on Dec. 4, 1973, discloses a system whereby changes are produced in an animal's systemic arterial blood pressure, cerebrospinal fluid pressure, and heart rate through the use of warm or cold water irrigation of the nasal mucosa of the animal. It is further disclosed that this irrigation of the nasal mucosa may selectively decrease the temperature of the animal's brain.

U.S. Pat. No. 5,372,608, issued to Bertrand L. Johnson on Dec. 13, 1994, discloses a cooling sleeve that is wrapped around an extremity of an individual. Ice water is circulated through a closed system, including internal piping means, to allow the cold fluid to cool the affected extremity.

The prior art does not disclose, either singularly or in combination, any of the embodiments of the invention herein.

### BRIEF SUMMARY OF THE INVENTION

The embodiments of the invention provide a method and apparatus for affecting the temperature of an area of the body. The embodiments of the invention may be utilized to alter the temperature of the blood traveling through the carotid artery of a person.

In one embodiment, the device generally comprises a container, a container cap connectable thereto, at least one inlet tube, at least one outlet tube, and a temperature-altering element contained in an operable configuration in relation to the container. The at least one inlet tube is positioned such that at least one end of the inlet tube is in proximity to the interior space of the container. The at least one outlet tube is positioned such that at least one end of the outlet tube is in proximity to the interior space of the container.

In one embodiment of a method of use the steps include the operable coupling of a temperature-altering element with a container; introducing fluid into the interior space of the container; altering the temperature of the fluid; and expelling the altered fluid toward a body surface.

In an alternative embodiment, a container cap may be removably attached to the container. Alternatively, the container and container cap may be integrally formed.

In an alternative embodiment, the container, container cap, at least one inlet tube, and at least one outlet tube are integrally formed.

In another alternative embodiment, the temperature-altering element is connected to the exterior of the container.

In another alternative embodiment, the container and container cap form a substantially airtight enclosure when they are in a closed configuration.

In another alternative embodiment, a positioning strap is utilized to position the container relative to a body.

In an exemplary embodiment, the container cap contains at least one orifice throughout which at least one outlet tube and at least one inlet tube traverse.

Other features, embodiments, and advantages of embodiments of the invention will be apparent from the following description, the accompanying drawings and the appended claims.

### BRIEF DESCRIPTION OF THE DRAWINGS

For a more complete understanding of the present invention and the advantages thereof, reference is now made to the following description, taken in conjunction with the accompanying drawings, in which:

FIG. 1 is a front perspective view of an embodiment of the invention.

FIG. 2 is a perspective view of the container of an embodiment of the invention.

FIG. 3 is a perspective top view of the container cap of an embodiment of the invention.

FIG. 4 is a perspective side view of the container cap of an embodiment of the invention.

FIG. 5 is a cross-sectional view of an embodiment of the invention in use.

FIG. 6 is a side perspective view of an alternative embodiment of the invention.

### DESCRIPTION OF THE INVENTION

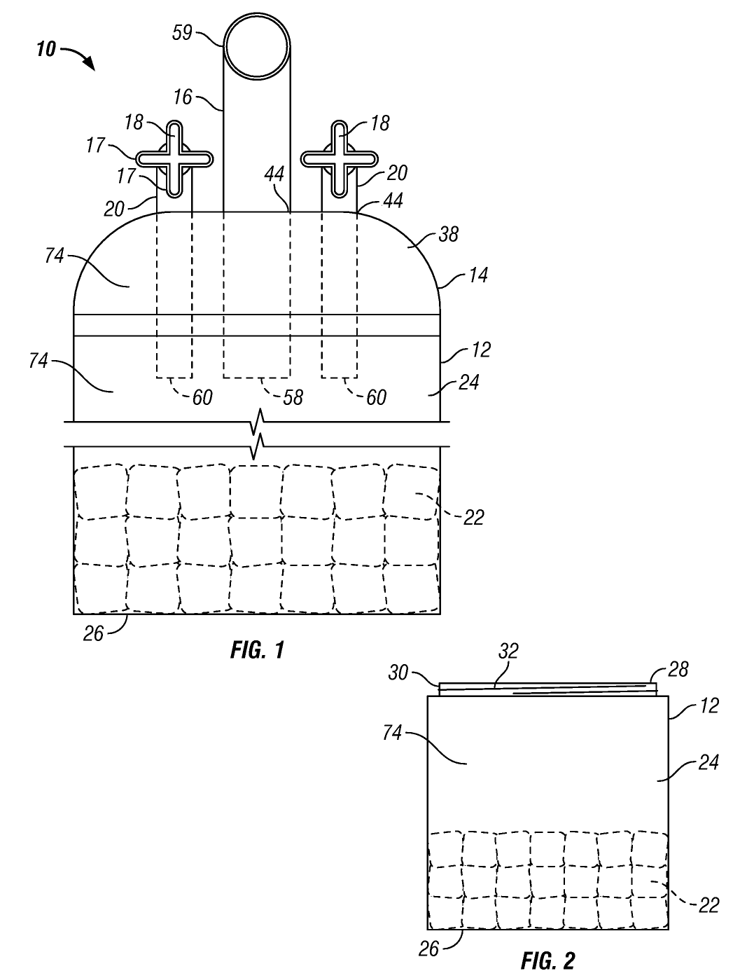

The embodiments of the invention and their advantages are best understood by referring to the drawings, like numerals being used for like and corresponding parts of the various drawings. FIGS. 1-5 show an embodiment of the invention.

Referring to FIGS. 1 and 2, an embodiment of the invention 10 depicted comprises at least one container 12, at least one corresponding container cap 14, at least one inlet tube 16, and at least one outlet tube 20.

The container 12, in an exemplary embodiment, is generally comprised of a body member 24 and a base member 26. The base member 26 is a generally circular, substantially flattened, disklike member. The body member 24 is generally annular in shape and extends radially. However, it is within the scope of the invention for the base member 26 and body member 24 to comprise any number of alternative shapes, such as cuboidal, spherical, spheroid, parallelepiped, polyhedron or any other desirable configuration. The body member 24 is fixedly attached to the base member 26; however, in an alternative embodiment, the body member 24 may be removably attached to the base member 26. The base member 26 is disposed distal the top of the container 28.

The container connection member 30 is generally located proximate the top of the container 28 and distal the base member 26. The outer diameter of the container connection member 30, as shown in this exemplary embodiment, is smaller than the outer diameter of the body member 24. In alternative embodiments the outer diameter of the container connection member 30 may be less than, equal to or greater than the outer diameter of the body member 24. In alternative embodiments the inner diameter of the container connection member 30 may be less than, equal to or greater than the inner diameter of the body member 24. At least one thread 32 is formed around the container connection member 30 for threadably connecting the container 12 to the container cap 14.

Referring to FIGS. 1, 2, 3, and 4, the container cap 14, in an exemplary embodiment, is generally comprised of a cap member 38 and a container cap connection member 36. The cap member 38, as shown, is generally arcuately formed; however, it is within the scope of the invention for the cap member 38 to comprise any number of alternative shapes, such as cuboidal, spherical, spheroid, parallelepiped, polyhedron or any other desirable configuration.

The cap member 38 contains a container cap connection member 36 proximate the base of the container cap 40. The container cap connection member 36 is distal the top of the container cap 42. The container cap connection member 36 is shown as generally annular in shape; however, the shape of the container cap connection member 36 will be dictated by the shape of the container connection member 30. It is contemplated that any number of shapes would perform the same functions as the general shapes disclosed herein for this exemplary embodiment, any one of the alternative shapes, as used in an embodiment of this invention, will retain the novelty and inventive inspiration expressed herein as utilized in that embodiment.

The container cap connection member 36 contains at least one corresponding thread 34 disposed proximate the base of the container top 40. The at least one corresponding thread 34 corresponds to the at least one thread 32 located on the container connection member 30. The at least one thread 32 and the at least one corresponding thread 34 allow for the connection of the container 12 and container cap 14.

A function of the container 12 and container cap 14 is to contain the temperature-altering element 22. Another function of the container 12 and container cap 14 is to allow for the movement of fluid 48 over and/or throughout the temperature-altering element 22. A further function of the container 12 and container cap 14 is to contain the fluid 48. A function of the connection members 30,36 is to fixedly or removably connect the container cap 14 and the container 12.

The container 12 and container cap 14 are connectable. This connection can either be in a fixedly connectable or removably connectable configuration. This connection may also be integral wherein the container 12 and container cap 14 are formed as one piece.

In an alternative embodiment, the container 12 and container cap 14 are substantially airtight when in a closed configuration. This airtight configuration may be achieved by the use of substantially airtight material proximate the closure area, with an extraneous substantially airtight sealing material, with an integrated substantially airtight sealing material or the like. The airtight configuration may also be achieved by the positioning of the container in relation to the positioning of the container cap or any combination of the aforementioned.

Referring to FIGS. 1, 3, and 4, at least one orifice 44 is located in the container cap 14. The inlet and outlet tubes 16,20 extend through the at least one orifice 44. The orifice 44 may be placed in any location that will allow fluid 48 to flow through inlet and outlet tubes 16,20 and over and/or through the temperature-altering element 22 and out of the container 12. The term fluid as used herein includes gases and/or liquids.

The exemplary embodiment shown depicts three orifices 44. The orifices 44 wherein the inlet tube 16 traverses has a slightly larger diameter than the orifice 44 containing the outlet tube 20. However, in alternate embodiments the diameters of the orifices 44 may be the same or may vary in any number of combinations. A purpose of the orifices 44 is the transmittal of the fluid 48 in to and out of the container 12. This function is achieved by the flow of the fluid 48 through the tubes 16,20 that provide ingress and egress through the at least one orifice 44.

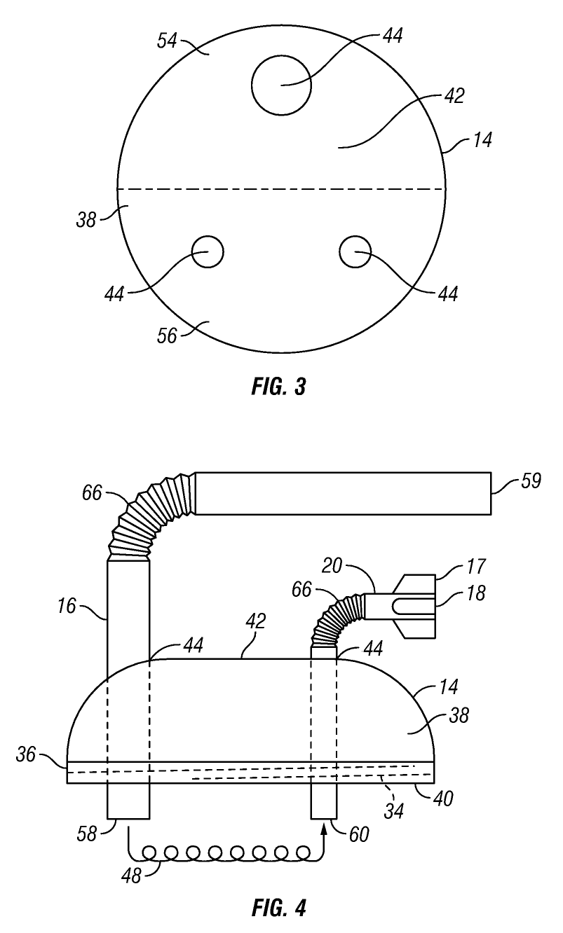

The exemplary embodiment shown in FIG. 3 depicts the orifice 44 coupled with the inlet tube 16 disposed within one hemisphere 54 of the container cap 14 and the orifices 44 coupled with the outlet tubes 20 disposed within the directly opposed hemisphere 56. Alternative embodiments may contain the orifices 44 in any number of configurations within the container cap 14. Examples of such alternative configurations include, but are not limited to, containing all of the orifices 44 within one hemisphere 54 or 56 (not shown), containing an inlet tube orifice 44 and an outlet tube orifice 44 in hemisphere 54 and additional inlet tube orifices 44 and additional outlet tube orifices 44 in the directly opposed hemisphere 56 (not shown), containing one orifice 44 with the inlet and outlet tubes 16,20 traversing therethrough, or any other desired configuration.

A purpose of the orifice 44 is to provide a passageway between the interior and exterior of the container 12 and container cap 14 combination for at least one inlet tube 16 and at least one outlet tube 20. A further purpose of the orifice 44 is to allow fluid 48 to flow into the interior space 74 of the container 12. A further purpose of the orifice 44 is to allow the altered fluid 50 to flow out of the interior space 74 of the container 12.

The inlet tube 16 is a generally elongated annular tube. The inlet tube 16 is of sufficient length that when installed the inlet insert end 58 is disposed within the interior space 74 of the container 12. The inlet intake end 59 of the inlet tube 16 extends out of the container cap 14 through orifice 44.

The outlet tube 20 is a generally elongated annular tube. The outlet tube 20 is also of sufficient length that when installed the outlet insert end 60 is disposed within the interior space 74 of the container 12. The outlet expelling end 18 of the outlet tube 20 extends out of the container cap 14 through orifice 44.

In an alternative embodiment, the at least one inlet tube 16 and at least one outlet tube 20 are each in a closely fitting configuration within at least one orifice 44. This configuration may be utilized in order to inhibit fluid 48 flow at the interface of the inlet tube 16 and outlet tube 20 with the container cap 14.

In an alternative embodiment, the at least one inlet tube 16 and at least one outlet tube 20 are each slideable within the at least one orifice 44. The position of the inlet 16 and outlet 20 tubes may then be manipulated. This manipulation may allow for the positioning of the insert ends 58,60 proximate to the temperature-altering element 22.

In one embodiment, the outlet expelling end 18 of the outlet tube 20 is in a relatively x-shaped configuration. According to an embodiment of the invention, the prongs 17 of the outlet expelling end 18 are substantially perpendicular to one another. This perpendicularity is not necessary as the relative nature of the x-shaped configuration allows for varying degrees of angularity. Angles other than right angles may also be utilized between the prongs 17. A purpose of the shape of the outlet expelling end 18 in this embodiment is to increase the velocity of the altered fluid 50 as it is expelled. This increase in velocity may aid in the dispersion of the altered fluid 50 across a greater surface area of the neck 72. Therefore, many embodiments may utilize any number of shapes in lieu of or in addition to an x-shape for the outlet expelling end 18 in order to effect this purpose. Alternatively, the outlet expelling end 18 may simply be composed of a generally annular opening similar to the outlet insert end 60 as shown or a similar surface that does not aid in expanding the surface area of the outlet expelling end 18.

The at least one outlet tube 20 may contain a flexible joint 66 intermediate the outlet insert end 60 and outlet expelling end 18. The flexible joint 66 allows for flexion of the outlet tube 20. This flexion aids in directing the flow of the altered fluid 50 by manipulating the positioning of the outlet expelling end 18. Similarly, the at least one inlet tube 16 may contain a flexible joint 66 intermediate the inlet insert end 58 and the inlet intake end 59. This flexible joint 66 also allows for the flexion of the inlet tube 16 which allowed for the manipulation of the position of the inlet intake end 59. Indirectly this flexion also allows for the manipulation of the insert ends 58,60 in relation to the inlet intake end 59 and/or outlet expelling end 18.

The at least one outlet tube 20 and/or the at least one inlet tube 16, in alternative embodiments, may be fixedly or flexibly formed in varying curved or straight orientations in order to conform to the requirements of the embodiment of the invention 10. Alternatively, the flexible material of the flexible joint 66, or some similar material that would allow the inlet tube 16 and the outlet tube 20 to be flexible, may encompass all or a majority of the respective tubes 16,20.

A purpose of the container 12 is to contain the temperature-altering element 22 in an operable configuration in relation to the container 12. In the exemplary embodiment shown, the body member 24 and base member 26 together provide a location for the temperature-altering element 22 to be placed in order to allow for fluid 48 to flow over and/or through the temperature-altering element 22 in order to affect the temperature of the fluid 48. This change in temperature of the fluid 48 then produces the altered fluid 50 that is released near or directly onto the skin of an individual in order to affect the temperature of that particular location. The altered fluid 50 is fluid 48 that has been either cooled or heated within the interior space 74.

The temperature-altering element 22 may include ice as shown or may include other substrates (not shown), such as a liquid, a block of ice, plastic containers filled with heated liquid or ice, chemicals that when activated become either hot or cold, as well as other types of hot or cold materials and substrates. This includes materials that affect the temperature of fluids 48 that would flow over and/or through the material. In an alternative embodiment, a liquid is placed into the container 12 and frozen. The use of cooling material or devices would cool the fluid 48 as it travels through the at least one inlet tube 16, through the interior space 74 of the container 12, and out of the at least one outlet tube 20. The use of heating material or devices would heat the fluid 48 as it travels through the at least one inlet tube 16, through the interior space 74 of the container 12, and out of the at least one outlet tube 20.

In an alternative embodiment, the temperature-altering element 22 may be placed in an operable configuration in relation to the container 12 by its 22 connection to the exterior surface of the container 12. The temperature-altering element 22 will act upon the container 12 in order to affect the temperature of the member 12 and in essence affect the temperature of the fluid 48 contained and/or introduced therein.

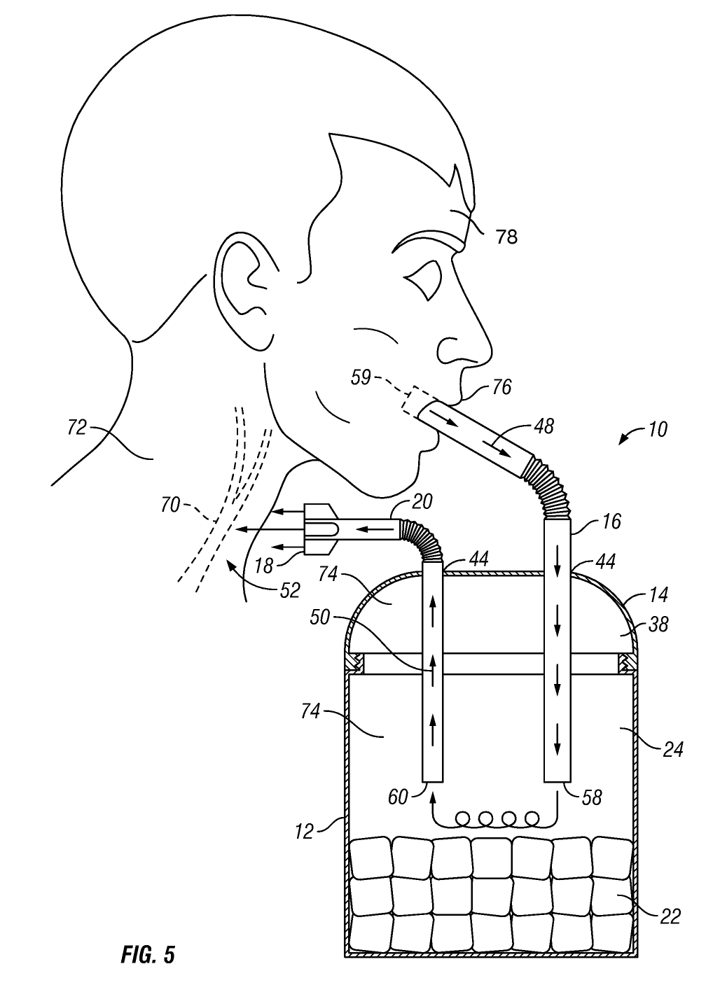

Referring to FIG. 5, the release of the altered fluid 50 may be directed towards the neck in the general location of the carotid artery 70. The altered fluid 50 may be directed at any location that is meant to be heated or cooled depending on the type of temperature-altering element 22 employed.

In the embodiment shown in FIG. 5, the container 12 contains a plurality of individual ice cubes as the temperature-altering element 22. The fluid 48 employed in this embodiment is air that is physically blown through the inlet tube 16 by a person 78 though their mouth 76 at the inlet intake end 59 of the inlet tube 16. However, the fluid 48 may be supplied by any known method including but not limited to the use of a fluid expelling device such as a motorized or hand-held pump, air compressor, or the like. The fluid 48 then flows through the interior of the inlet tube 16 and enters the interior space 74 of the container 12 through the inlet insert end 58. The fluid 48 then circulates over and/or through the temperature-altering element 22 and in doing so the fluid 48 is cooled below the temperature point in which it entered the interior space 74 of the container 12 to become the altered fluid 50. The altering of the fluid 48 occurs by the cooling of the fluid from contact with the temperature-altering element 22, in the embodiment depicted element 22 is ice, and its environment. The altered fluid 50 then travels through the outlet insert end 60. The altered fluid 50 proceeds throughout the interior of the outlet tube 20 and through the outlet expelling end 18.

The outlet expelling end 18 of the outlet tube 20 may be positioned so that it faces the approximate area of a person's neck 72 that the carotid artery 70 traverses 52. The altered fluid 50 is expelled from the outlet expelling end 18 of the outlet tube 20 and affects an area 52 of the person's neck 72 at or about where the carotid artery 70 extends. This contact of the altered cooled fluid 50, aids the cooling of the blood flowing through the carotid artery thereby allowing cooled blood to flow to the person's brain. The outlet tubes 20 may be positioned in any location that the user wishes to expel the altered fluid 50 toward.

This method may also be utilized with a temperature-altering element 22 that is meant to heat the fluid 48. In an alternative embodiment, the temperature-altering element 22 is comprised of warm liquid, a heating coil, or some other substrate or apparatus that would be used to increase the temperature of the fluid 48. Rather than cool the area of the body that the at least one outlet tube 20 is positioned to face, the altered fluid 50 would warm the area. This method would be useful in many applications in which the body temperature of an individual drops below that which is normal or drops to a point where body function is affected. In this instance the alternative embodiment utilizing a heat inducing temperature-altering element 22 may be used to warm up the blood flowing through the carotid artery in order to warm up the individual.

Multiple containers 12 and container caps 14 may be utilized at once either in conjunction or individually.

In an alternative embodiment, the container 12 and container cap 14 are integrally formed (not shown). In a further alternative embodiment, the container 12, container cap 14, at least one inlet tube 16 and at least one outlet tube 20 are integrally formed.

In an alternative embodiment, positioning straps (not shown) may be operationally connected to the container 12 and/or container cap 14. The straps may be any commercially available straps available now or in the future. The positioning straps may be positionable to at least one position in relation to the body. The positioning straps may position the container 12 at a desired location in order to use the invention and/or perform the method contained herein.

A method of use of an embodiment of the invention may include the steps of operably coupling a temperature-altering element 22 with a container 12; introducing fluid 48 into the interior space 74 of the container 12; altering the temperature of the fluid 48; and expelling the altered fluid 50 toward a body surface 72.

In an alternative embodiment, a connecting step is also performed. The connecting step comprises connecting the container cap 14 to the container 12.

A further alternative embodiment utilizes a gas as the fluid 48 to be altered.

Alternatively, the introducing step may comprise the expulsion of air 48 from a mouth 76 through at least one inlet tube 16. The inlet tube 16 being in fluid communication with the interior space of the container 12.

In an alternative embodiment, a manipulating step is performed. At least one outlet tube 20 is manipulated such that the outlet expelling end 18 is directed toward a body surface area.

In another alternative embodiment, a securing step is performed whereby the container 12 is secured to the body. The container 12 may be secured to the body by a positioning strap (not shown).

Alternatively, the method may utilize any of the embodiments disclosed herein or that would naturally flow from the information contained herein in relation to utilizing embodiments of the invention. Further, the ordering of the aforementioned steps is somewhat arbitrary. By way of example, the securing step may be performed prior or subsequent to virtually all other listed steps.

When ice made from water is utilized as the temperature-altering element 22, the resulting water that melts during operation may be given to the individual to aid in comforting or to further cool the individual.

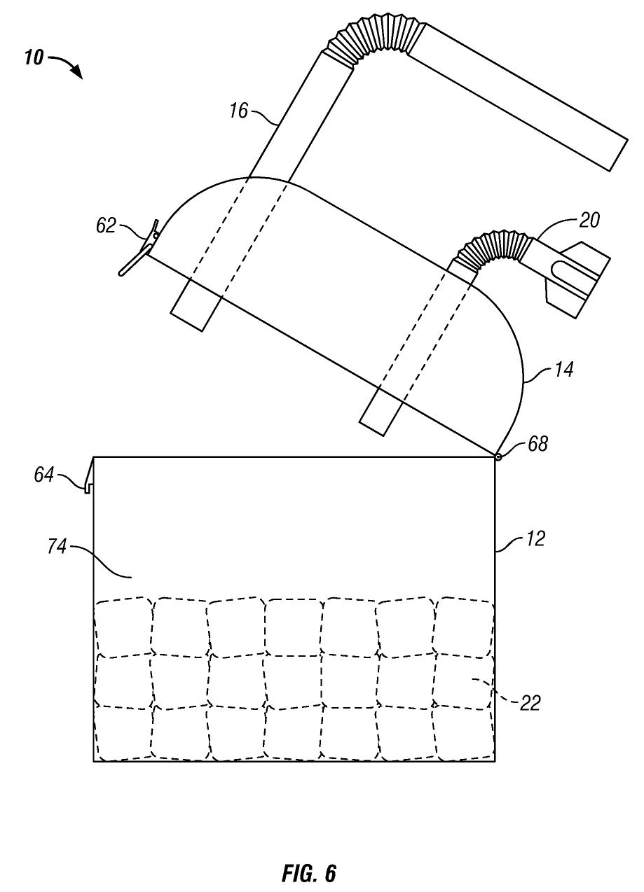

Referring to FIG. 6, in an alternative embodiment the container 12 and the container cap 14 are fixedly or removably attached at a pivot junction 68. Latching members 62,64 are fixedly or removably attached to the embodiment with lower latching member 64 attached to the container 12 and a corresponding upper latching member 62 attached to the container cap 14. The pivot point 68 is utilized to bring the latching members 62,64 into close proximity to connect the container cap 14 to the container 12. Once the latching members 62,64 are released, the container cap 14 may be pivotally lifted away from the container 12 at the pivot point 68.

In an alternative embodiment the container cap 14 and the container 12 are slidingly secured (not shown). In this alternative embodiment, the container cap connection member 36 contains an outer diameter that is only slightly smaller than the inner diameter of the container connection member 30. Due to this slight variation the container cap connection member 36 may slidingly engage onto the container connection member 30 in order to connect the container cap 14 with the container 12. Alternatively, the outer diameter of the container connection member 30 may be slightly smaller than the inner diameter of the container cap connection member 36.

There are a number of mechanisms and methods that may be utilized to connect the container 12 to the container cap 14 including, but not limited to, snap-top connections, compressive gaskets, gluing, compressive latch mechanisms, compressive spring mechanisms, snap couplings, bayonet couplings, zipping, hook and loop fasteners, bolting mechanisms, elastic bands, wire, sliding mechanisms, plugs, compressive clips, twist lid, epoxying, or any other mechanism or method that would allow for the connection.

In an alternative embodiment, a leak proof seal (not shown) is utilized to prevent the leakage of fluid from between the container connection member 30 and the container cap connection member 36.

In a further alternative embodiment, the container 12 is at least partially insulated with a suitable insulating material. In an alternative embodiment, the container cap 14 and the container 12 are each at least partially insulated with a suitable insulating material.

Alternatively, the container 12 and/or container cap 14 may be constructed of an insulating material or at least partially constructed of an insulating material.

The insulation may be positioned such that a portion of the container 12 and or container cap 14 is left uninsulated. The uninsulated portion may then be placed proximate the body in order to either cool or heat the area proximate to the uninsulated portion via the uninsulated area.

The foregoing description of example embodiments of the invention illustrates exemplary embodiments thereof. Various changes may be made in the details of the illustrated construction within the scope of the appended claims without departing from the true spirit of the invention. The present invention should only be limited by the claims and their equivalents.

What is claimed is:

1. A temperature modifying apparatus, comprising:
a container having an interior space;
a container cap connectable to said container;
a temperature-altering element positioned in an operable configuration in relation to said container;
at least one inlet tube having an inlet insert end;
said inlet insert end structured for insertion into said interior space;
at least one outlet tube having an outlet insert end; and
said outlet insert end structured for insertion into said interior space.

2. The device of claim 1, wherein said temperature-altering element is contained within said interior space.

3. The device of claim 1, wherein said temperature-altering element is connected to the exterior of said container.

4. The device of claim 1, wherein said temperature-altering element is ice.

5. The device of claim 1, wherein said container and said container cap form a substantially airtight enclosure when they are in a closed configuration.

6. The device of claim 1, further comprising:
at least one positioning strap; and
said at least one positioning strap operationally connectable to said container and positionable relative to a body.

7. The device of claim 1, further comprising:
said container cap having at least one orifice;
said at least one inlet tube extending through said at least one orifice such that said inlet insert end is positioned within said interior space; and
said at least one outlet tube extending through said at least one orifice such that said outlet insert end is positioned within said interior space.

8. The device of claim 7, further comprising:
at least one flexible joint member operably integrated into said at least one inlet tube whereby said at least one inlet tube is manipulable; and
said at least one flexible joint member operably integrated into said at least one outlet tube whereby said at least one outlet tube is manipulable.

9. The device of claim 7, wherein said at least one inlet tube and said at least one outlet tube are each in a closely fitting configuration within said at least one orifice in order to inhibit fluid flow at their interface with said container cap.

10. The device of claim 7, wherein said at least one inlet tube and said at least one outlet tube are each slideable within said at least one orifice in order to manipulate the positioning thereof.

11. The device of claim 7, further comprising:
said at least one inlet tube having an inlet intake end; and
said inlet intake end structured to allow a human to physically blow air therethrough.

12. The device of claim 7, further comprising:
said at least one inlet tube having an inlet intake end; and
a fluid expelling device operationally coupled to said inlet intake end in order to expel fluid therethrough.

13. The device of claim 1, wherein said container is at least partially insulated.

14. A method of modifying the temperature of a body area, said method comprising:
a coupling step comprising operably coupling a temperature-altering element with a container;
an introducing step comprising introducing a fluid into an interior space of said container;
an altering step comprising altering the temperature of said fluid; and
an expelling step comprising expelling said altered fluid toward a body surface.

15. The method of claim 14, further comprising a connecting step comprising connecting said container to a container cap.

16. The method of claim 14, wherein said fluid is a gas.

17. The method of claim 14, wherein said introducing step comprises expulsion of air from a person's mouth through at least one inlet tube in fluid communication with said interior space.

18. The method of claim 14, further comprising a manipulating step comprising manipulating at least one outlet tube having an outlet expelling end such that said outlet expelling end is directed toward said body surface.

19. The method of claim 14, further comprising a securing step comprising securing said container to a body.

20. A temperature modifying apparatus, comprising:
a container having an interior space;
a container cap connectable to said container;
a temperature-altering element contained within said interior space;
at least one inlet tube having an inlet insert end;
at least one outlet tube having an outlet insert end;
said container cap having at least one orifice;
said at least one inlet tube extending through said at least one orifice such that said inlet insert end is positioned within said interior space;
said at least one outlet tube extending through said at least one orifice such that said outlet insert end is positioned within said interior space;
at least one flexible joint member;
said at least one flexible joint member operably integrated into said at least one inlet tube whereby said at least one inlet tube is manipulable;
said at least one flexible joint member operably integrated into said at least one outlet tube whereby said at least one outlet tube is manipulable; and
said at least one outlet tube having an outlet expelling end wherein said outlet expelling end is in a relatively x-shaped configuration.
